(19) United States
(12) Patent Application Publication
(10) Pub. No.: US 2024/0335568 A1
(43) Pub. Date: Oct. 10, 2024

(54) RADIOPAQUE POLYSACCHARIDE HYDROGELS AND METHODS OF MAKING THE SAME

(71) Applicant: Boston Scientific Scimed, Inc., Maple Grove, MN (US)

(72) Inventors: Yen-Hao Hsu, Shrewsbury, MA (US); Joseph Thomas Delaney, JR., Minneapolis, MN (US); Cristian Parisi, Boston, MA (US); Heather Cirka, Hudson, MA (US); Alexis Compton, Wrentham, MA (US)

(73) Assignee: Boston Scientific Scimed, Inc., Maple Grove, MN (US)

(21) Appl. No.: 18/627,837

(22) Filed: Apr. 5, 2024

Related U.S. Application Data
(60) Provisional application No. 63/457,922, filed on Apr. 7, 2023.

(51) Int. Cl. A61K 49/04

(52) U.S. Cl. CPC A61K 49/0438; A61K 49/0457; A61K 49/0495

(57) ABSTRACT

In some aspects, the present disclosure pertains to radiopaque hydrogel compositions that comprise a radiopaque polysaccharide that comprises a plurality of radiopaque moieties that are covalently linked to a carboxylic-acid-containing polysaccharide along a backbone of the carboxylic-acid-containing polysaccharide. In some aspects, the present disclosure pertains to radiopaque hydrogel compositions that comprise a carboxylate-anion-containing polysaccharide that is ionically crosslinked by multivalent cations. In other aspects, the present disclosure pertains to kits that contain such radiopaque hydrogel compositions, to methods of treatment that comprise administering such radiopaque hydrogel compositions to patients, and to methods of making such radiopaque hydrogel compositions.

### CROSS-REFERENCE TO RELATED APPLICATIONS

This application claims the benefit of U.S. Provisional Patent Application Ser. No. 63/457,922 filed on Apr. 7, 2023, the disclosure of which is incorporated herein by reference.

### FIELD

The present disclosure relates to radiopaque polysaccharide hydrogels and to methods of making and using such hydrogels, among other aspects. The radiopaque polysaccharide hydrogels of the present disclosure are useful in various biomedical applications.

### BACKGROUND

Non-animal stabilized hyaluronic acid (NASHA) solutions form physically crosslinked hydrogels, suitable for injections and as fillers, with good biocompatibility. One hyaluronic acid-based hydrogel, available as Barrigel®, is used to minimize radiation-associated side effects during prostate cancer treatment by creating the space in between the prostate and rectum. Barrigel®, however, does not have significant radiopacity.

There is a continuing need in the biomedical arts for hydrogels that are radiopaque, for methods of making and using such radiopaque hydrogels, and for systems for forming such radiopaque hydrogels, among other needs.

### SUMMARY

In some aspects, the present disclosure pertains to radiopaque hydrogel compositions that comprise a radiopaque polysaccharide that comprises a plurality of radiopaque moieties that are covalently linked to a carboxylic-acid-containing polysaccharide along a backbone of the carboxylic-acid-containing polysaccharide.

In some embodiments, the radiopaque moieties are covalently linked to the carboxylic-acid-containing polysaccharide through amide or ester bonds.

In some embodiments, which can be used in conjunction with the above aspects and embodiments, the carboxylic-acid-containing polysaccharide comprises one or more uronic acid species selected from galacturonic acid, glucuronic acid, and iduronic acid.

In some embodiments, which can be used in conjunction with the above aspects and embodiments, the carboxylic-acid-containing polysaccharide is selected from hyaluronic acid, alginic acid, pectin, agaropectin, carrageenan, gellan gum, gum arabic, guar gum, xanthan gum, and carboxymethyl cellulose.

In some embodiments, which can be used in conjunction with the above aspects and embodiments, the carboxylic-acid-containing polysaccharide has a number average molecular weight ranging from 1 kDa to 100 kDa.

In some embodiments, which can be used in conjunction with the above aspects and embodiments, the radiopaque moieties comprise iodinated moieties.

In some embodiments, which can be used in conjunction with the above aspects and embodiments, the radiopaque moieties comprise one or more iodinated aromatic groups.

In some embodiments, which can be used in conjunction with the above aspects and embodiments, the radiopaque moieties comprise one or more iodinated aromatic groups that are substituted with one or more hydrophilic groups.

In some embodiments, which can be used in conjunction with the above aspects and embodiments, the radiopaque moieties comprise iodinated moieties that comprise residues of primary-amine-substituted iodinated compounds. In some of these embodiments, the primary-amine-substituted iodinated compounds are selected from iodinated amino acids and iodinated amino acid esters. In some of the embodiments, the primary-amine-substituted iodinated compounds comprise an aromatic group that is substituted with a primary amine group and one or more iodine groups and, optionally, one or more hydrophilic groups.

In some embodiments, which can be used in conjunction with the above aspects and embodiments, the radiopaque moieties comprise beta-, gamma-, delta- or epsilon-amino acid residues positioned between iodinated moieties and the backbone of the carboxylic-acid-containing polysaccharide.

In some aspects, the present disclosure pertains to radiopaque hydrogel compositions that comprise a carboxylate-anion-containing polysaccharide that is ionically crosslinked by multivalent cations.

In some embodiments, the multivalent cations are multivalent radiopaque cations selected from Ba²⁺, Bi³⁺, Gd³⁺, Gd³⁺, Ta⁵⁺, W⁶⁺ and Au⁴⁺.

In some embodiments, which can be used in conjunction with the above aspects and embodiments, the radiopaque hydrogel compositions comprise an iodinated salt.

In some embodiments, which can be used in conjunction with the above aspects and embodiments, the radiopaque hydrogel compositions have a radiopacity that is greater than 100 Hounsfield units.

In some embodiments, which can be used in conjunction with the above aspects and embodiments, the radiopaque hydrogel compositions further comprising a therapeutic agent. For example, the therapeutic agent may be selected from antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, and STING (stimulator of interferon genes) agonists.

In some embodiments, which can be used in conjunction with the above aspects and embodiments, radiopaque hydrogel compositions further comprise a colorant.

In some embodiments, which can be used in conjunction with the above aspects and embodiments, the radiopaque hydrogel compositions are selected from injectable compositions and orally ingestible compositions.

In some embodiments, which can be used in conjunction with the above aspects and embodiments, the radiopaque polysaccharide hydrogel compositions are provided in a reservoir, such as a syringe barrel or a vial.

In other aspects the present disclosure pertains to methods of treatment that comprise administering to a subject a radiopaque polysaccharide hydrogel composition in accordance with any of the above aspects and embodiments.

In other aspects the present disclosure pertains to methods of making a radiopaque polysaccharide compound comprising reacting a primary amine group of a primary-amine-substituted iodinated compound with carboxylic acid groups of a carboxylic-acid-containing polysaccharide in an amide coupling reaction.

In still other aspects, the present disclosure pertains to a method of making a radiopaque polysaccharide compound comprising (a) reacting in a first amide coupling reaction a primary amine group of a primary-amine-substituted iodinated compound with a carboxylic acid group of an amino acid in which the primary amine group of the amino acid is protected, (b) deprotecting the protected primary amine group of the product of step (a); and (c) reacting in a second amide coupling reaction the deprotected primary amine of the product of step (b) with carboxylic acid groups of a carboxylic-acid-containing polysaccharide.

The above and other aspects, embodiments, features and benefits of the present disclosure will be readily apparent from the following detailed description.

### BRIEF DESCRIPTION OF THE DRAWINGS

FIG. 1 schematically illustrates a method of making a radiopaque hyaluronic acid hydrogel in accordance with an embodiment of the present disclosure.

FIG. 2 schematically illustrates a method of making a radiopaque alginic acid hydrogel in accordance with an embodiment of the present disclosure.

FIG. 3 schematically illustrates a method of making a radiopaque hyaluronic acid hydrogel in accordance with another embodiment of the present disclosure.

FIG. 4 schematically illustrates a method of making a radiopaque alginic acid hydrogel in accordance with another embodiment of the present disclosure.

FIG. 5 schematically illustrates a method of making a radiopaque hyaluronic acid hydrogel in accordance with yet another embodiment of the present disclosure.

### DETAILED DESCRIPTION

In various embodiments, the radiopaque polysaccharide hydrogel compositions of the present disclosure comprise a radiopaque polysaccharide in which radiopaque atoms are linked to the polysaccharide to provide the compositions with radiopacity.

In various embodiments, the radiopaque polysaccharide hydrogel compositions of the present disclosure comprise a radiopaque polysaccharide that comprises a polysaccharide moiety and a plurality of radiopaque moieties that comprise radiopaque atoms, which radiopaque moieties are linked to the polysaccharide moiety.

As detailed below, the radiopaque atoms may be covalently or ionically linked to the polysaccharide moiety. Examples of radiopaque atoms include I, Br, Bi, Ba, Gd, Ta, Zn, Au, Pt, Ca, and W.

In some embodiments, the radiopaque polysaccharides of the present disclosure comprise a polysaccharide moiety and a plurality of radiopaque moieties that are covalently linked along a backbone of the polysaccharide moiety. In some of these embodiments, the radiopaque moieties are covalently linked to the polysaccharide moiety through amide groups or ester groups. In some of these embodiments, the radiopaque moieties are iodinated moieties. However, it should be noted that, while iodine atoms are exemplified, other radiopaque atoms including bromine atoms may be employed in the radiopaque moieties.

In various embodiments, the polysaccharide moiety is derived from a carboxylic-acid-containing polysaccharide. Carboxylic-acid-containing polysaccharides include any polysaccharide that comprises carboxylic acid groups, such as polysaccharides that contain one or more uronic acid species, such as galacturonic acid, glucuronic acid and/or iduronic acid. Particular examples of carboxylic-acid-containing polysaccharides include alginic acid, hyaluronic acid, pectin, agaropectin, carrageenan, gellan gum, gum arabic, guar gum, xanthan gum, and carboxymethyl cellulose moieties. In embodiments where the carboxylic-acid-containing polysaccharide is hyaluronic acid, the carboxylic-acid-containing polysaccharide may be non-animal stabilized hyaluronic acid. Alginic acid is a linear copolymer containing (1,4)-linked β-D-mannuronate (M) and α-L-guluronate (G) residues. In embodiments where the carboxylic-acid-containing polysaccharide is alginic acid, the alginic acid is preferably highly purified and has a low content of the M residues to limit any immunogenic response. A lower content of M residues is also expected to promote better mechanical properties such as enhanced strength and ductility. Similarly, a higher content of the G residues in the alginic acid should result in a higher elastic modulus and lower swelling when introduced into aqueous environments. In some embodiments, the polysaccharide moiety is derived from a carboxylic-acid-containing polysaccharide having a number average molecular weight ranging from 1 kDa to 8000 kDa, for example ranging anywhere from 1 kDa to 2.5 kDa to 5 kDa to 10 kDa to 25 kDa to 50 kDa to 100 kDa to 250 kDa to 500 kDa to 1000 kDa to 2000 kDa to 8000 kDa (in other words, ranging between any two of the preceding numerical values).

Examples of iodinated moieties of the present disclosure include those that comprise one, two, or more iodinated aromatic groups (also referred to as iodo-aromatic groups). Examples of iodinated aromatic groups include iodine-substituted monocyclic aromatic groups and iodine-substituted multicyclic aromatic groups, such as iodine-substituted phenyl groups and iodine-substituted naphthyl groups. The aromatic groups may be substituted with one, two, three, four, five, six or more iodine atoms. In various embodiments, the aromatic groups may be further substituted with one or more hydrophilic groups, for example, one, two, three, four, five, six or more hydrophilic groups. The hydrophilic groups may comprise, for example, one or more of the following groups: hydroxyl groups, hydroxyalkyl groups (e.g., hydroxyalkyl groups containing one carbon, two carbons, three carbons, four carbons, etc.), ester groups (e.g., ester groups containing two carbons, three carbons, four carbons, five carbons, six carbons, etc.), or carboxyl groups. The hydrophilic groups may be linked to the aromatic group directly or through any suitable linking moiety, which may be selected, for example, from alkyl groups (e.g., alkyl groups containing one carbon, two carbons, three carbons, four carbons, etc.), amide groups, amine groups, ether groups, ester groups, or carbonate groups, among others.

Examples of iodinated moieties include residues of iodinated amino acids or residues of iodinated amino acid esters, for example, C₁-C₅-alkyl esters of iodinated amino acids, typically, methyl esters of iodinated amino acids, as described in further detail below.

Specific examples of iodinated moieties include iodinated moieties which are linked to the polysaccharide through an iodinated aromatic group, such as the following, among many others:

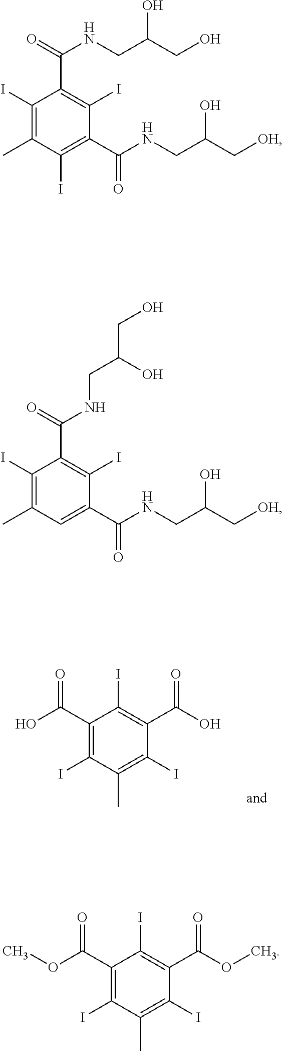 [SMILES: Cc1c(I)c(C(=O)NCC(O)CO)c(I)c(C(=O)NCC(O)CO)c1I ; Cc1cc(C(=O)NCC(O)CO)c(I)c(C(=O)NCC(O)CO)c1I ; Cc1c(I)c(C(=O)O)c(I)c(C(=O)O)c1I ; COC(=O)c1c(I)c(C)c(I)c(C(=O)OC)c1I]

Specific examples of iodinated moieties further include iodinated amino acid residues, including the following, among many others:

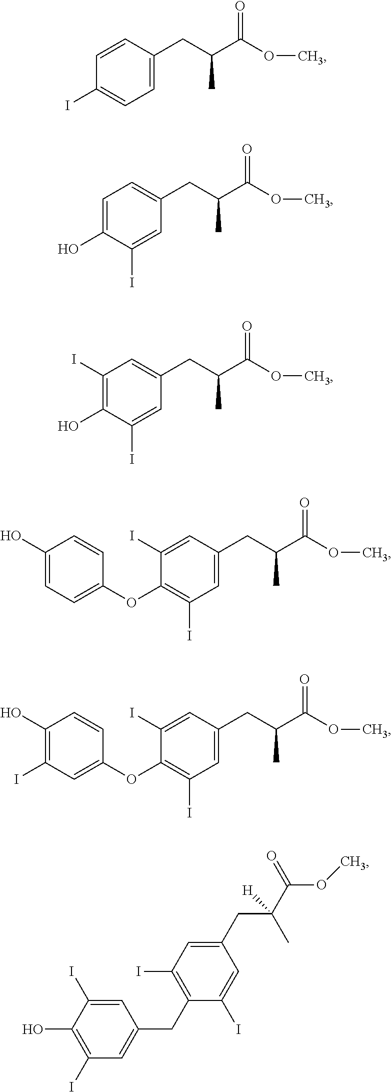 [SMILES: COC(=O)[C@@H](C)Cc1ccc(I)cc1 ; COC(=O)[C@@H](C)Cc1ccc(O)c(I)c1 ; COC(=O)[C@@H](C)Cc1cc(I)c(O)c(I)c1 ; COC(=O)[C@@H](C)Cc1cc(I)c(Oc2ccc(O)cc2)c(I)c1 ; COC(=O)[C@@H](C)Cc1cc(I)c(Oc2ccc(O)c(I)c2)c(I)c1 ; COC(=O)[C@@H](C)Cc1cc(I)c(Cc2cc(I)c(O)c(I)c2)c(I)c1]

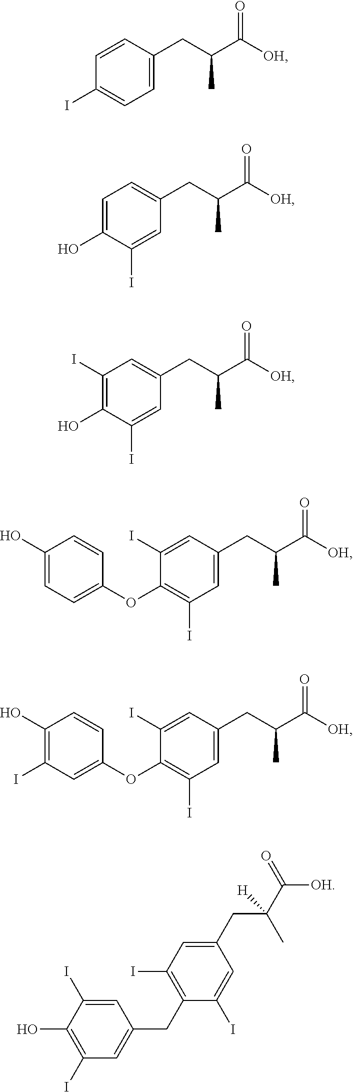 [SMILES: C[C@@H](Cc1ccc(I)cc1)C(=O)O ; C[C@@H](Cc1ccc(O)c(I)c1)C(=O)O ; C[C@@H](Cc1cc(I)c(O)c(I)c1)C(=O)O ; C[C@@H](Cc1cc(I)c(Oc2ccc(O)cc2)c(I)c1)C(=O)O ; C[C@@H](Cc1cc(I)c(Oc2ccc(O)c(I)c2)c(I)c1)C(=O)O ; C[C@@H](Cc1cc(I)c(Cc2cc(I)c(O)c(I)c2)c(I)c1)C(=O)O]

Such iodinated moieties may be linked to a polysaccharide moiety, for example, through an amide group.

In various embodiments, the radiopaque polysaccharides of the present disclosure may comprise a residue of a carboxylic-acid-containing polysaccharide and a plurality of residues of a primary-amine-substituted iodinated compound. The primary-amine-substituted iodinated compound residues may be linked along a backbone of the carboxylic-acid-containing polysaccharide residue through amide linkages.

In various embodiments, a primary amine group of a primary-amine-substituted iodinated compound may be reacted with carboxylic acid groups of a carboxylic-acid-containing polysaccharide in an amide coupling reaction to form a radiopaque polysaccharide. Such an amide coupling reaction may be performed using a suitable coupling reagent, for instance, a carbodiimide coupling reagent such as 1-ethyl-3-(3-dimethylaminopropyl) carbodiimide (EDC) or a salt thereof, such as 1-ethyl-3-(3-dimethylaminopropyl) carbodiimide hydrochloride (EDC·HCl). In various embodiments, between 1% and 100% (for example, ranging from 1% to 2.5% to 5% to 10% to 25% to 50% to 75% to 90% to 95% to 97.5% to 99% to 100%) of the carboxylic acid groups of the carboxylic-acid-containing polysaccharide may be converted to amide groups.

Examples of primary-amine-substituted iodinated compounds include 5-amino-N,N′-bis (2,3-dihydroxypropyl)-2,4,6-triiodoisophthalamide (also known as Iohexol related compound J),

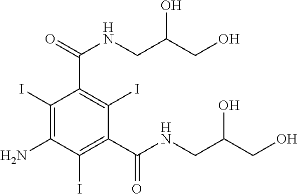 [SMILES: Nc1c(I)c(C(=O)NCC(O)CO)c(I)c(C(=O)NCC(O)CO)c1I]

(CAS #76801-93-9), 5-amino-N1,N3-bis (2,3-dihydroxypropyl)-2,4-diiodoisophthalamide,

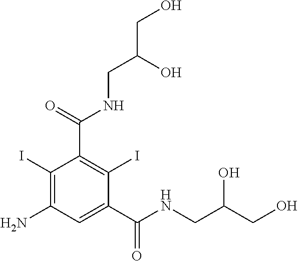 [SMILES: Nc1cc(C(=O)NCC(O)CO)c(I)c(C(=O)NCC(O)CO)c1I]

(CAS #1215856-35-1), and dimethyl 5-amino-2,4,6-triiodo-1,3-benzenedicarboxylate,

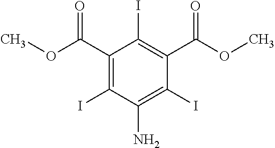

(CAS #154921 Nov. 6), among others.

Examples of primary-amine-substituted iodinated compounds further include iodinated amino acid esters, for example, C₁-C₅-alkyl esters of iodinated amino acids, preferably methyl esters of iodinated amino acids. Particular examples include C₁-C₅-alkyl esters of any of the iodinated amino acids described below. After coupling, the C₁-C₆-alkyl ester may be converted into the corresponding carboxylic acid, if desired.

As used herein, an “amino acid” is an organic compound that contain an amino group (—NH₂), a carboxylic acid group (—COOH), and a side group that is specific to each amino acid. Depending on the surrounding pH, the amino group may be positively charged (—NH₃⁺) and/or the carboxylic acid group may be negatively charged (—COO⁻). An iodinated amino acid is an amino acid in which the side group contains one or more iodine atoms.

In various embodiments, the side group of the iodinated amino acid comprises one, two, or more iodinated aromatic groups. Examples of iodinated aromatic groups include iodine-substituted monocyclic aromatic groups and iodine-substituted multicyclic aromatic groups, such as iodine-substituted phenyl groups and iodine-substituted naphthyl groups. The aromatic groups may be substituted with one, two, three, four, five, six or more iodine atoms. In various embodiments, the aromatic groups may be further substituted with one or more hydrophilic groups, for example, one, two, three, four, five, six or more hydrophilic groups. The hydrophilic groups may comprise, for example, one or more of the following groups: hydroxyl groups, hydroxyalkyl groups (e.g., hydroxyalkyl groups containing one carbon, two carbons, three carbons, four carbons, etc.), ester groups (e.g., ester groups containing two carbons, three carbons, four carbons, five carbons, six carbons, etc.), or carboxyl groups. The hydrophilic groups may be linked to the aromatic group directly or through any suitable linking moiety, which may be selected, for example, from alkyl groups (e.g., alkyl groups containing one carbon, two carbons, three carbons, four carbons, etc.), amide groups, amine groups, ether groups, ester groups, or carbonate groups, among others.

Examples of iodinated amino acid esters include iodinated alpha-amino acid esters, iodinated beta-amino acid esters, iodinated gamma-amino acid esters, iodinated delta-amino acid esters, and iodinated epsilon-amino acid esters.

Specific examples of iodinated amino acid esters include the following: iodo-phenylalanine methyl ester.

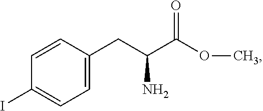 [SMILES: COC(=O)[C@@H](N)Cc1ccc(I)cc1]

monoiodotyrosine methyl ester,

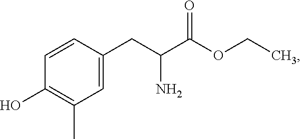 [SMILES: CCOC(=O)C(N)Cc1ccc(O)c(C)c1]

diiodotyrosine methyl ester,

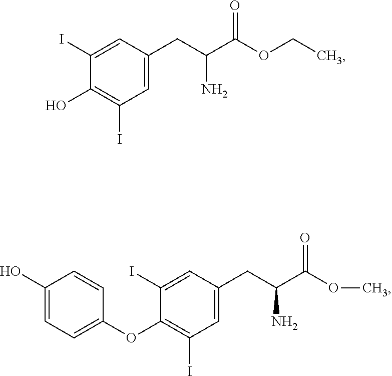 [SMILES: CCOC(=O)C(N)Cc1cc(I)c(O)c(I)c1 ; COC(=O)[C@@H](N)Cc1cc(I)c(Oc2ccc(O)cc2)c(I)c1]

triiodothyronine methyl ester, also known as T3 methyl ester.

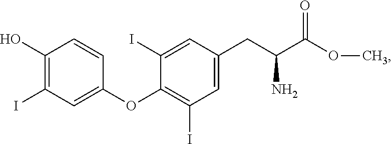 [SMILES: COC(=O)[C@@H](N)Cc1cc(I)c(Oc2ccc(O)c(I)c2)c(I)c1]

tetraiodothyronine methyl ester, also known as thyroxine methyl ester or T4 methyl ester,

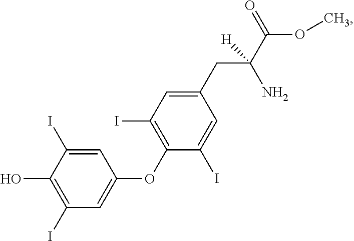 [SMILES: COC(=O)[C@@H](N)Cc1cc(I)c(Oc2cc(I)c(O)c(I)c2)c(I)c1]

iodo-phenylalanine methyl ester, and 6-iodo-L-DOPA methyl ester, among others. Although methyl esters are shown, higher alkyl esters may be employed.

As previously noted, the radiopaque polysaccharide hydrogel compositions of the present disclosure may be formed by an amide coupling reaction between a carboxylic-acid containing polysaccharide, such as one of those described above, among others, and a primary-amine-substituted iodinated compound, such as one of those described above, among others. Such an amide coupling reaction may be performed using a suitable coupling reagent, for instance, a carbodiimide coupling reagent. Significantly, radiopacity can be tunable based on loading of iodinated residues by varying functionalization density.

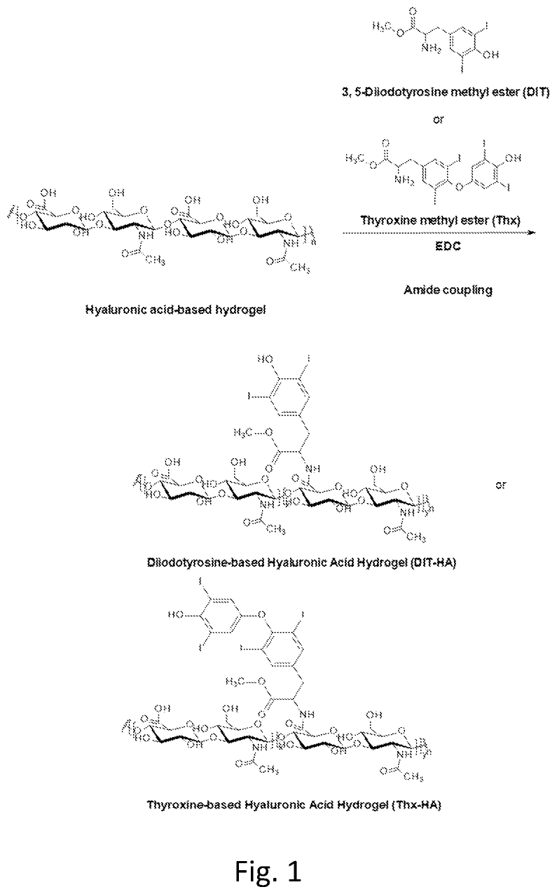

In a particular example shown in FIG. 1, hyaluronic acid is employed as the carboxylic-acid containing polysaccharide and either 3,5-diiodotyrosine methyl ester, CAS #76318-50-8 or thyroxine methyl ester, CAS #76318-50-8, is employed as the primary-amine-substituted iodinated compound. 1-ethyl-3-(3-dimethylaminopropyl) carbodiimide (EDC) is used as a coupling reagent.

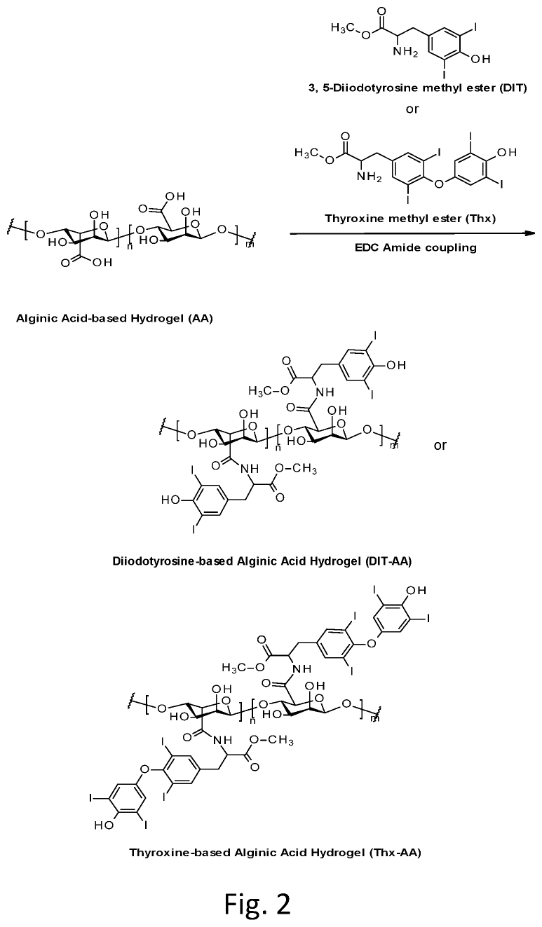

In another example shown in FIG. 2, alginic acid is employed as the carboxylic-acid containing polysaccharide and either 3,5-diiodotyrosine methyl ester or thyroxine methyl ester is employed as the primary-amine-substituted iodinated compound. 1-ethyl-3-(3-dimethylaminopropyl) carbodiimide (EDC) is used as a coupling reagent.

Several of the primary-amine-substituted iodinated compounds described above, including Iohexol related compound J,

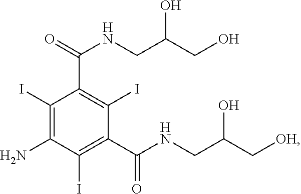 [SMILES: Nc1c(I)c(C(=O)NCC(O)CO)c(I)c(C(=O)NCC(O)CO)c1I]

have a primary amine group that is directly linked to an iodinated aromatic group, which may result in steric hindrance when the primary amine group is reacted with a carboxylic acid group of a carboxylic-acid containing polysaccharide to form an amide bond. In such embodiments, it may be desirable to provide a spacer moiety between the polysaccharide moiety and the iodinated moiety.

In certain of these embodiments, the spacer moiety may be formed using a suitable non-iodinated amino acid compound for example, selected from beta amino acids such as 3-aminopropanoic acid (also known as beta-alanine),

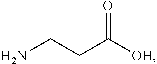

gamma amino acids such as 4-aminobutanoic acid (also known as gamma-aminobutyric acid, or GABA),

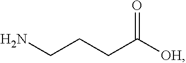

delta amino acids such as 5-aminopentanoic acid.

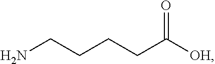

epsilon amino acids, and so forth.

In some of these embodiments, the primary amine of the amino acid is first protected using a suitable protection agent, such as di-tert-butyl decarbonate. Then, the carboxylic acid group of the protected amino acid is reacted with the primary amine of a primary-amine-substituted iodinated compound, typically in the presence of a suitable amine coupling agent, such as a carbodiimide coupling agent, to form an amide bond. After amide bond formation, the primary amine group of the amino acid residue is deprotected. Subsequently, the deprotected primary amine group of the amino acid residue is reacted with carboxylic acid groups of a carboxylic-acid containing polysaccharide in an amide coupling reaction, typically in the presence of a suitable amine coupling agent such as a carbodiimide coupling agent, thereby forming an amide bond.

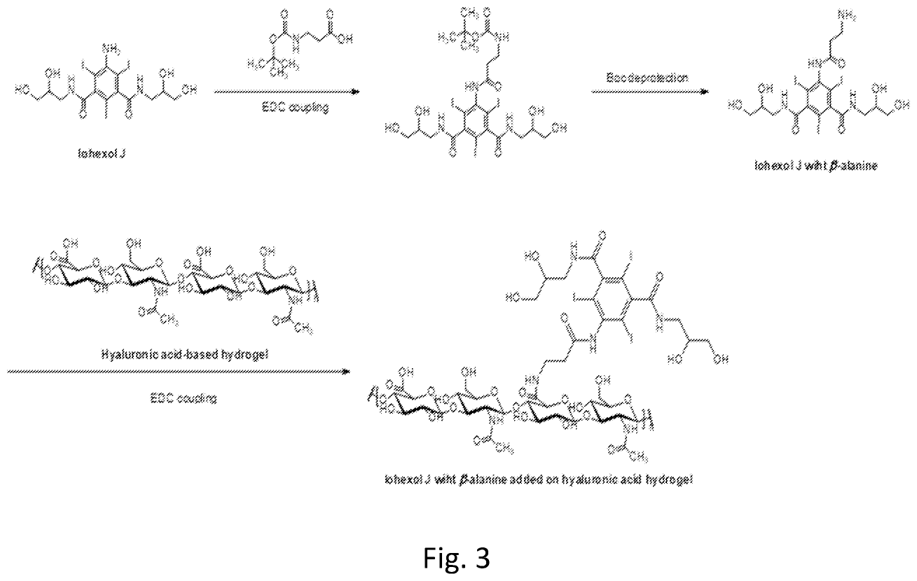

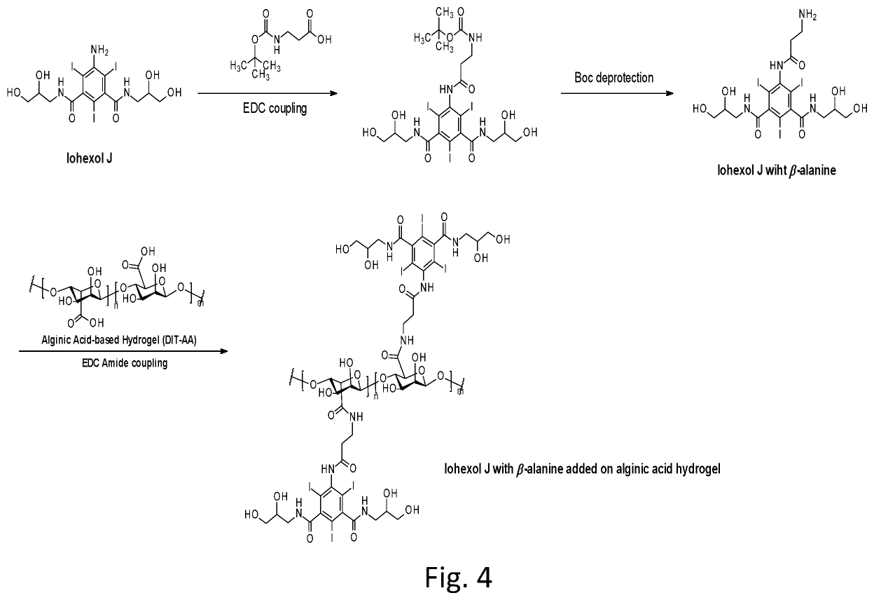

In a particular example shown in FIG. 3, beta-alanine is used to create a spacer between a hyaluronic acid moiety and an Iohexol J moiety. In a first step shown in FIG. 3, the primary amine group of Iohexol J is reacted with the carboxylic acid group of beta-alanine in which the primary amine group has been protected. Specifically, the primary amine group of Iohexol J is reacted with the carboxylic acid group of t-Boc-protected beta-alanine in an amide coupling reaction (e.g., using a carbodiimide coupling reagent such as EDC) to form an amide bond. In a second step, the primary amine group of the beta-alanine is deprotected, for example, by exposing the t-Boc protected product of the first step to acidic conditions using an acid such as trifluoroacetic acid or hydrochloric acid. Then, the deprotected primary amine group of the product of the second step is reacted with carboxylic acid groups of hyaluronic acid in an amide coupling reaction, using EDC as coupling reagent, thereby forming a radiopaque polysaccharide. An analogous reaction sequence is illustrated in FIG. 4, except that alginic acid, rather than hyaluronic acid, is used as used to form the radiopaque polysaccharide.

As previously noted, in some embodiments, radiopaque moieties are covalently linked to the polysaccharide moiety through ester groups. For example, in various embodiments, a hydroxyl group of a hydroxyl-substituted iodinated compound may be reacted with carboxylic acid groups of a carboxylic-acid-containing polysaccharide in an ester coupling reaction to form a radiopaque polysaccharide. Particular examples of hydroxyl-substituted iodinated compounds include iodixanol (CAS #92339 Nov. 2),

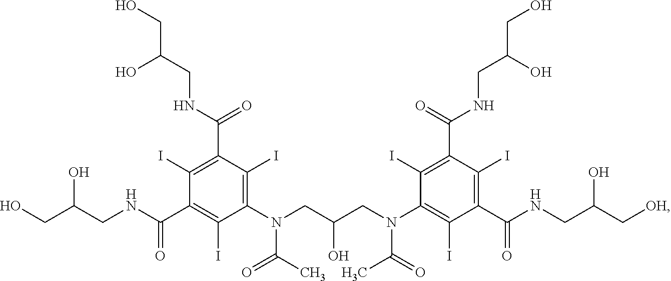 [SMILES: CC(=O)N(CC(O)CN(C(C)=O)c1c(I)c(C(=O)NCC(O)CO)c(I)c(C(=O)NCC(O)CO)c1I)c1c(I)c(C(=O)NCC(O)CO)c(I)c(C(=O)NCC(O)CO)c1I]

and its derivatives, and Ioversol (CAS #87771-40-2),

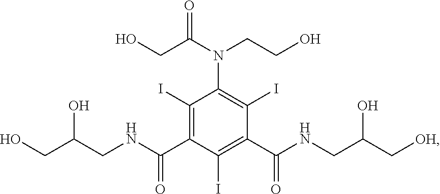 [SMILES: O=C(NCC(O)CO)c1c(I)c(C(=O)NCC(O)CO)c(I)c(N(CCO)C(=O)CO)c1I]

and its derivatives. In addition to providing a source of radiopacity, these compounds can also act as crosslinking agents, since there are multiple hydroxyl groups on the structures.

Such an ester coupling reaction may be performed using a suitable coupling reagent, for instance, a carbodiimide coupling reagent such as 1-ethyl-3-(3-dimethylaminopropyl) carbodiimide (EDC) or a salt thereof, such as 1-ethyl-3-(3-dimethylaminopropyl) carbodiimide hydrochloride (EDC·HCl). In various embodiments, between 1% and 100% (for example, ranging from 1% to 2.5% to 5% to 10% to 25% to 50% to 75% to 90% to 95% to 97.5% to 99% to 100%) of the carboxylic acid groups of the carboxylic-acid-containing polysaccharide may be converted to ester groups.

Using the above and other techniques, iodinated polysaccharide compounds can be provided in which an iodinated moiety is linked to a polysaccharide moiety through an amino acid residue. Examples of amino acid residues include beta amino acid residues such as a 3-aminopropanoic acid residue, gamma amino acid residues, such as a 4-aminobutanoic acid residue, delta amino acid residues, such as a 5-aminopentanoic acid residue, and epsilon amino acid residues, among others.

As previously indicated, in various embodiments, the radiopaque polysaccharide hydrogel compositions of the present disclosure include those that include a radiopaque polysaccharide that comprises a polysaccharide moiety and a plurality of ionically-linked radiopaque atoms. In some embodiments, the radiopaque atoms are in the form of multi-valent radiopaque cations that are used to ionically crosslink negatively charged groups of anionic polysaccharides. Anionic polysaccharides include deprotonated versions of carboxylic-acid-containing polysaccharides, which are also referred to herein as carboxylate-anion-containing polysaccharides. The negatively charged carboxylate anion groups of the carboxylate-anion-containing polysaccharides can form ionic bonds (also referred to as electrostatic bonds) with the multivalent cations, which also act to ionically crosslink the carboxylate-anion-containing polysaccharides. Examples of carboxylate-anion-containing polysaccharides include deprotonated versions of the carboxylic-acid-containing polysaccharides set forth above. Examples of multi-valent radiopaque cations include Ba²⁺, Bi³⁺, Gd³⁺, Gd³⁺, Ta⁵⁺, W⁶⁺ and Au⁴⁺, among others.

In some embodiments, such ionically crosslinked polysaccharides can be formed by first deprotonating a carboxylic-acid-containing polysaccharide such as one of those described above. For example, a carboxylic-acid-containing polysaccharide can be treated with a base such as sodium hydroxide or potassium hydroxide to form a carboxylate-anion-containing polysaccharide salt, preferably a monovalent cationic salt, for example, an alkali metal salt such as a lithium (Li⁺), sodium (Na⁺), potassium (K⁺), rubidium (Rb⁺), cesium (Cs⁺) or francium (Fr⁺) salt. Then the monovalent cations are exchanged with multivalent cations, for example, by mixing an aqueous solution of BaCl₂, an aqueous solution of BiCl₃, an aqueous solution of GdCl₃, an aqueous solution of TaCl₅, an aqueous solution of WCl₆, or an aqueous solution of AuCl₄ with the carboxylate-anion-containing polysaccharide salt forming a polycluster by coordination.

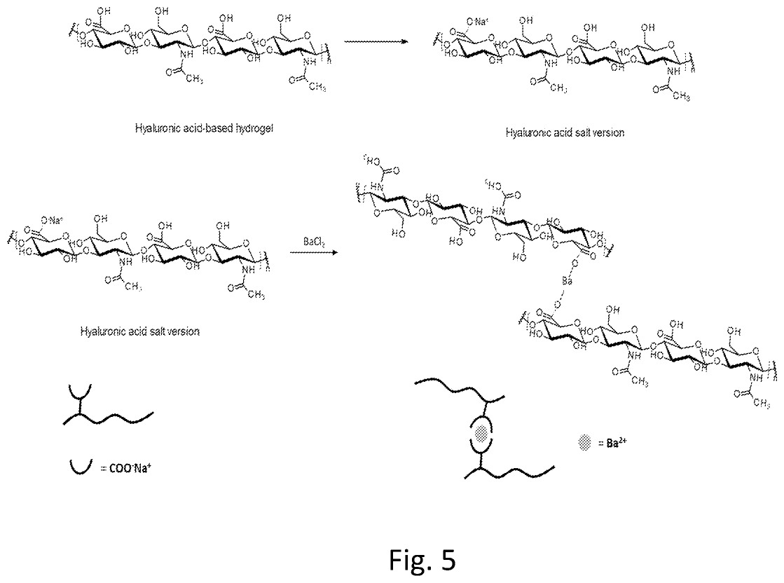

In a particular embodiment shown in FIG. 5, ionically crosslinked hyaluronic acid is formed by first deprotonating hyaluronic acid by treating hyaluronic acid with sodium hydroxide to form a hyaluronic acid sodium salt. Then, the monovalent sodium ions of the hyaluronic acid sodium salt are exchanged with multivalent barium cations by mixing hyaluronic acid sodium salt with an aqueous solution of BaCl₂. Analogous procedures may be performed with BiCl₃, GdCl₃, TaCl₅, WCl₆, or AuCl₄ as noted above.

In further embodiments, the radiopaque polysaccharide hydrogel compositions of the present disclosure include those that include a radiopaque polysaccharide that comprises a polysaccharide moiety, a multi-valent cation that is used to ionically crosslink negatively charged groups of anionic polysaccharides, and a radiopaque anion such as a bromide or iodide anion. Anionic polysaccharides include deprotonated versions of carboxylic-acid-containing polysaccharides, which are also referred to herein as carboxylate-anion-containing polysaccharides. The negatively charged carboxylate anion groups of the carboxylate-anion-containing polysaccharides can form ionic bonds with the multivalent cations, which also act to ionically crosslink the carboxylate-anion-containing polysaccharides. Examples of carboxylate-anion-containing polysaccharides include deprotonated versions of the carboxylic-acid-containing polysaccharides set forth above. Examples of multi-valent radiopaque cations include Be²⁺, Mg²⁺ and Ca²⁺, as well as multi-valent radiopaque cations such as Ba²⁺, Bi³⁺, Gd³⁺, Gd³⁺, Ta⁵⁺, W⁶⁺ and Au⁴⁺ discussed above, among others. Such radiopaque polysaccharide hydrogel compositions may be formed, for example, by first deprotonating a carboxylic-acid-containing polysaccharide such as one of those described above. For example, a carboxylic-acid-containing polysaccharide can be treated with a base such as sodium hydroxide or potassium hydroxide to form a carboxylate-anion-containing polysaccharide salt, preferably a monovalent cationic salt, for example, an alkali metal salt such as a lithium (Li⁺), sodium (Na⁺), potassium (K⁺), rubidium (Rb⁺), cesium (Cs⁺) or francium (Fr⁺) salt. Then the monovalent cations are exchanged with multivalent cations, for example, by mixing an aqueous solution of Bel₂, an aqueous solution of Mgl₂, an aqueous solution of Cal₂, an aqueous solution of Bal₂, an aqueous solution of Bil₃, an aqueous solution of GdI₃, an aqueous solution of Tals, an aqueous solution of WI₆, or an aqueous solution of Aul₄ with the carboxylate-anion-containing polysaccharide salt forming a polycluster by coordination.

In various embodiments, the radiopaque polysaccharide hydrogel compositions of the present disclosure are visible under fluoroscopy. In various embodiments, such radiopaque polysaccharide hydrogel compositions have a radiopacity that is greater than 100 Hounsfield units (HU), beneficially anywhere ranging from 100 HU to 250 HU to 500 HU to 750 HU to 1000 HU or more. Such radiopaque polysaccharide hydrogel compositions can be used in a wide variety of biomedical applications, including medical devices, oral dosage forms such as preformed hydrogels in pill or pouch form, injectable implants, and pharmaceutical compositions.

In addition to a radiopaque polysaccharide as described above, radiopaque polysaccharide hydrogel compositions in accordance with the present disclosure may contain additional agents. Examples of additional agents include therapeutic agents. Examples of therapeutic agents include antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, and STING (stimulator of interferon genes) agonists among others.

Examples of additional agents also include colorants such as brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents also include imaging agents in addition to the radiopaque atoms that are present in the radiopaque polysaccharide. Such imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd^{(III)}, Mn^{(ID)}, Fe^{(III)} and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) radiocontrast agents, such as those based on the clinically important isotope ^{99m}Tc, as well as other gamma emitters such as ¹²³I, ¹²⁵I, ¹³¹I, ¹¹¹In, ⁵⁷Co, ¹⁵³Sm, ¹³³Xe, ⁵¹Cr, ^{81m}Kr, ²⁰¹Tl, ⁶⁷Ga, and ⁷⁵Se, among others, (e) positron emitters, such as ¹⁸F, ¹¹C, ¹³N, ¹⁵O, and ⁶⁸Ga, among others, may be employed to yield functionalized radiotracer coatings, and (f) contrast agents for use in connection with near-infrared (NIR) imaging, which can be selected to impart near-infrared fluorescence to the coatings of the present disclosure, allowing for deep tissue imaging and device marking, for instance, NIR-sensitive nanoparticles such as gold nanoshells, carbon nanotubes (e.g., nanotubes derivatized with hydroxyl or carboxyl groups, for instance, partially oxidized carbon nanotubes), dye-containing nanoparticles, such as dye-doped nanofibers and dye-encapsulating nanoparticles, and semiconductor quantum dots, among others. NIR-sensitive dyes include cyanine dyes, squaraines, phthalocyanines, porphyrin derivatives and borondipyrromethane (BODIPY) analogs, among others.

In various embodiments, kits are provided that include one or more delivery devices for delivering the radiopaque polysaccharide hydrogel compositions to a subject. Such systems may include one or more of the following: a syringe barrel, which may or may not contain a radiopaque polysaccharide composition as described herein; a vial, which may or may not contain a radiopaque polysaccharide composition as described here; a needle; a flexible tube (e.g., adapted to fluidly connect the needle to the syringe), an injectable liquid such as water for injection, normal saline or phosphate buffered saline. Whether supplied in a syringe, vial, or other reservoir, the radiopaque polysaccharide composition may be provided in dry form (e.g., powder form) or in a form that is ready for injection, such as an injectable hydrogel form.

The radiopaque polysaccharide hydrogel compositions described herein can be used for a number of purposes.

For example, radiopaque polysaccharide hydrogel compositions can be injected to provide spacing between tissues, radiopaque polysaccharide hydrogel compositions can be injected (e.g., in the form of blebs) to provide fiducial markers, radiopaque polysaccharide hydrogel compositions can be injected for tissue augmentation or regeneration, radiopaque polysaccharide hydrogel compositions can be injected as a filler or replacement for soft tissue, radiopaque polysaccharide hydrogel compositions can be injected to provide mechanical support for compromised tissue, radiopaque polysaccharide hydrogel compositions be injected as a scaffold, and/or radiopaque polysaccharide hydrogel compositions can be injected as a carrier of therapeutic agents in the treatment of diseases and cancers and the repair and regeneration of tissue, among other uses. In other examples, radiopaque polysaccharide hydrogel compositions can be ingested to provide gastrointestinal imaging.

After administration, the compositions of the present disclosure can be imaged using a suitable imaging technique. Typically, the imaging techniques is an x-ray-based imaging technique, such as computerized tomography or X-ray fluoroscopy.

As seen from the above, the radiopaque polysaccharide hydrogel compositions of the present disclosure may be used in a variety of medical procedures, including the following, among others: a procedure to implant a fiducial marker comprising a radiopaque polysaccharide hydrogel composition, a procedure to implant a tissue regeneration scaffold comprising a radiopaque polysaccharide hydrogel composition, a procedure to implant a tissue support comprising a radiopaque polysaccharide hydrogel composition, a procedure to implant a tissue bulking agent comprising a radiopaque polysaccharide hydrogel composition, a procedure to implant a therapeutic-agent-containing depot comprising a radiopaque polysaccharide hydrogel composition, a tissue augmentation procedure comprising implanting a radiopaque polysaccharide hydrogel composition, a procedure to introduce a radiopaque polysaccharide hydrogel composition between a first tissue and a second tissue to space the first tissue from the second tissue or a procedure for diagnostic imaging purposes.

The polysaccharide hydrogel compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intra-vitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production, or ingestion to aid in diagnostic imaging.

As previously indicated, radiopaque polysaccharide hydrogel compositions in accordance with the present disclosure include compositions for therapeutic agent release (e.g., by including one or more therapeutic agents in a matrix of the crosslinked hydrogel) and implants (e.g., compositions for use as tissue markers, compositions that act as spacers to reduce side effects of off-target radiation therapy, cosmetic compositions, etc.), as well as lubricious compositions for medical applications.

1. A radiopaque hydrogel composition comprising a radiopaque polysaccharide that comprises a plurality of radiopaque moieties that are covalently linked to a carboxylic-acid-containing polysaccharide along a backbone of the carboxylic-acid-containing polysaccharide.

2. The radiopaque hydrogel composition of claim 1, wherein the radiopaque moieties are covalently linked to the carboxylic-acid-containing polysaccharide through amide or ester bonds.

3. The radiopaque hydrogel composition of claim 1, wherein the carboxylic-acid-containing polysaccharide comprises one or more uronic acid species selected from galacturonic acid, glucuronic acid, and iduronic acid.

4. The radiopaque hydrogel composition of claim 1, wherein the carboxylic-acid-containing polysaccharide is selected from hyaluronic acid, alginic acid, pectin, agaropectin, carrageenan, gellan gum, gum arabic, guar gum, xanthan gum, and carboxymethyl cellulose.

5. The radiopaque hydrogel composition of claim 1, wherein the radiopaque moieties comprise iodinated moieties.

6. The radiopaque hydrogel composition of claim 5, wherein the iodinated moieties comprise one or more iodinated aromatic groups.

7. The radiopaque hydrogel composition of claim 6, wherein the iodinated aromatic groups are substituted with one or more hydrophilic groups.

8. The radiopaque hydrogel composition of claim 5, wherein the iodinated moieties comprise residues of primary-amine-substituted iodinated compounds.

9. The radiopaque hydrogel composition of claim 8, wherein the primary-amine-substituted iodinated compounds are selected from iodinated amino acids and iodinated amino acid esters.

10. The radiopaque hydrogel composition of claim 8, wherein the primary-amine-substituted iodinated compounds comprise an aromatic group that is substituted with a primary amine group and one or more iodine groups.

11. The radiopaque hydrogel composition of claim 10, wherein the aromatic group is further substituted with one or more hydrophilic groups.

12. The radiopaque hydrogel composition of claim 5, further comprising a beta-, gamma-, delta-, or epsilon-amino acid residue positioned between the iodinated moieties and the backbone of the carboxylic-acid-containing polysaccharide.

13. The radiopaque hydrogel composition of claim 1, having a radiopacity that is greater than 100 Hounsfield units.

14. The radiopaque hydrogel composition of claim 1, wherein the radiopaque hydrogel composition is an injectable composition or an orally ingestible composition.

15. A method of treatment comprising administering to a subject a radiopaque polysaccharide hydrogel composition in accordance with claim 1.

16. A radiopaque hydrogel composition comprising a carboxylate-anion-containing polysaccharide that is ionically crosslinked by multivalent cations.

17. The radiopaque hydrogel composition of claim 16, wherein the multivalent cations are multivalent radiopaque cations selected from Ba²⁺, Bi³⁺, Gd³⁺, Gd³⁺, Ta⁵⁺, W⁶⁺ and Au⁴⁺.

18. The radiopaque hydrogel composition of claim 16, wherein the radiopaque hydrogel composition comprises an iodinated salt.

19. A kit comprising a reservoir and a radiopaque polysaccharide hydrogel composition disposed in the reservoir, the radiopaque polysaccharide hydrogel composition comprising (a) a radiopaque polysaccharide that comprises a plurality of radiopaque moieties that are covalently linked to a carboxylic-acid-containing polysaccharide along a backbone of the carboxylic-acid-containing polysaccharide or (b) a radiopaque hydrogel composition comprising a carboxylate-anion-containing polysaccharide that is ionically crosslinked by multivalent cations.

20. The kit of claim 15, wherein the reservoir is a syringe barrel or a vial.
